(19) United States
(12) Patent Application Publication
(10) Pub. No.: US 2010/0298669 A1
(43) Pub. Date: Nov. 25, 2010

(54) BAN-SENSOR RADIO COMMUNICATION DEVICE AND METHOD

(75) Inventor: Ichirou IDA, Kawasaki (JP)

Correspondence Address: Fujitsu Patent Center;Fujitsu Management Services of America, Inc., 2318 Mill Road, Suite 1010, Alexandria, VA, 22314, US

(73) Assignee: FUJITSU LIMITED, Kawasaki-shi (JP)

(21) Appl. No.: 12/781,302

(22) Filed: May 17, 2010

(30) Foreign Application Priority Data
May 21, 2009 (JP) 2009-123259

(51) Int. Cl. A61B 5/07

(52) U.S. Cl. 600/302

(57) ABSTRACT

A radio communication device for communicating sensor information includes an electromagnetic wave communication unit using electromagnetic waves using the air as a propagation path, an ultrasonic communication unit using ultrasonic waves using an inside of a living body as a propagation path, and a midair/living-body switching control unit for switching between a communication performed by the electromagnetic wave communication unit and a communication performed by the ultrasonic wave communication unit.

### CROSS-REFERENCE TO RELATED APPLICATION

This application is based upon and claims the benefit of priority of prior Japanese Patent application No. 2009-123259, filed on May 21, 2009, the entire contents of which are incorporated herein by reference.

### FIELD

The embodiments discussed herein are related to the communication technique of sensor information for a body area network.

### BACKGROUND

A sensor node provided with a sensing device and a radio communication device is disposed on a living body such as a human body or the like, and data such as body heat or the like is always measured. Thus, an application for improving the efficiency of examinations in medical facilities or reinforcing the health care provided in non-medical facilities is considered. In this case, a network of sensor nodes on a living body is sometimes called a “BAN (body area network)”.

Japanese laid-open Patent Publication Nos. 2000-49656, 2003-163644, 2001-144662, 2001-094516 and 2007-301160 disclose techniques related to the technique disclosed by this application.

### SUMMARY

A radio communication device for communicating sensor information includes: an electromagnetic wave communication unit using electromagnetic waves using the air as a propagation path; an ultrasonic communication unit using ultrasonic waves using the inside and/or the surface of a living body as a propagation path; a midair/living-body switching control unit for switching between a communication performed by the electromagnetic wave communication unit and a communication performed by the ultrasonic wave communication unit.

A radio communication method for communicating sensor information includes: switching between a communication performed by an electromagnetic wave communication unit using the air as a propagation path and a communication performed by an ultrasonic wave communication unit using a living body as a propagation path.

The object and advantages of the invention will be realized and attained by the elements and combinations particularly pointed out in the claims.

It is to be understood that both the foregoing general description and the following detailed description are exemplary and explanatory and are not restrictive of the invention as claimed.

### BRIEF DESCRIPTION OF DRAWINGS

FIG. 1 is a configuration of the preferred embodiment of a sensor network system on a living body.

FIG. 2 explains the basic operation of a communication using electromagnetic or ultrasonic waves, according to the preferred embodiment.

FIG. 3 is a configuration example of a sensor node.

FIG. 4 explains the operation of the preferred embodiment in the case where a communication via midair propagation using electromagnetic waves is conducted.

FIG. 5 explains the operation of the preferred embodiment in the case where a communication via intra-living-body propagation using ultrasonic waves is conducted.

FIG. 6 is an operational flowchart illustrating a midair electromagnetic wave/intra-living-body ultrasonic wave switching control algorithm (No. 1).

FIG. 7 is an operational flowchart illustrating a midair electromagnetic wave/intra-living-body ultrasonic wave switching control algorithm performed when high security is required (No. 2).

FIG. 8 explains the operation of the preferred embodiment in the case where high security is required.

FIG. 9 explains the operation of the preferred embodiment in the case where it is connected to a fixed network or the like.

FIG. 10 explains the preferred embodiment using an implant (built-into-body) type sensor.

FIG. 11 illustrates a sensor network technique for communicating using the air as a propagation path by an antenna.

FIG. 12 illustrates a sensor network technique for communicating using a living body surface as a propagation path by electrodes.

FIG. 13 is one example of the time fluctuation of received power due to fading.

### DESCRIPTION OF EMBODIMENTS

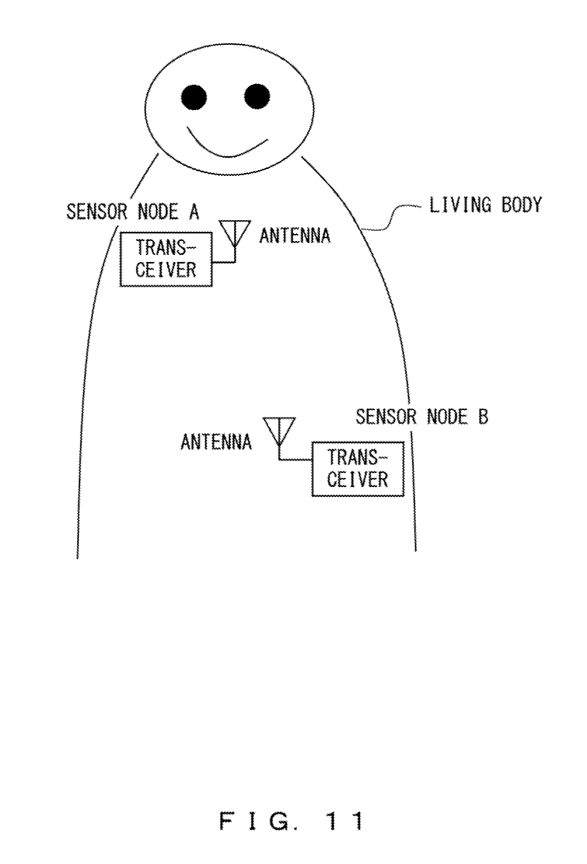

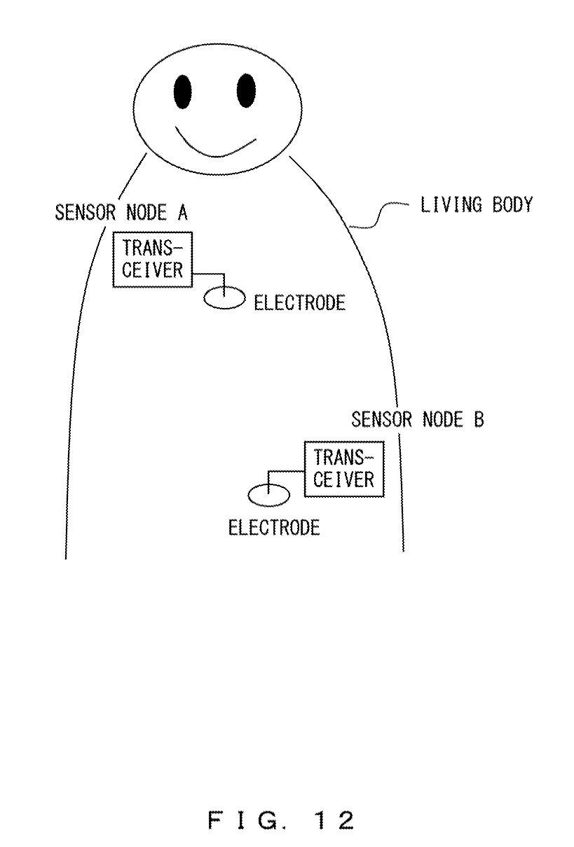

FIGS. 11 and 12 illustrate an antenna system for BAN communications. As a communication antenna in a BAN, a communication antenna using electromagnetic waves propagating in the air and a communication antenna using ultrasonic waves propagating on a living body surface are mainly studied. FIG. 11 is a configuration of an antenna system using electromagnetic waves propagating in the air. FIG. 12 is a configuration of an antenna system using ultrasonic waves propagating on a living body surface. Usually, the frequency in use of an antenna system using electromagnetic waves propagating in the air is several 100 MHz through several GHz, while the frequency in use of an antenna system using ultrasonic waves propagating on a living body surface is 10 MHz or less.

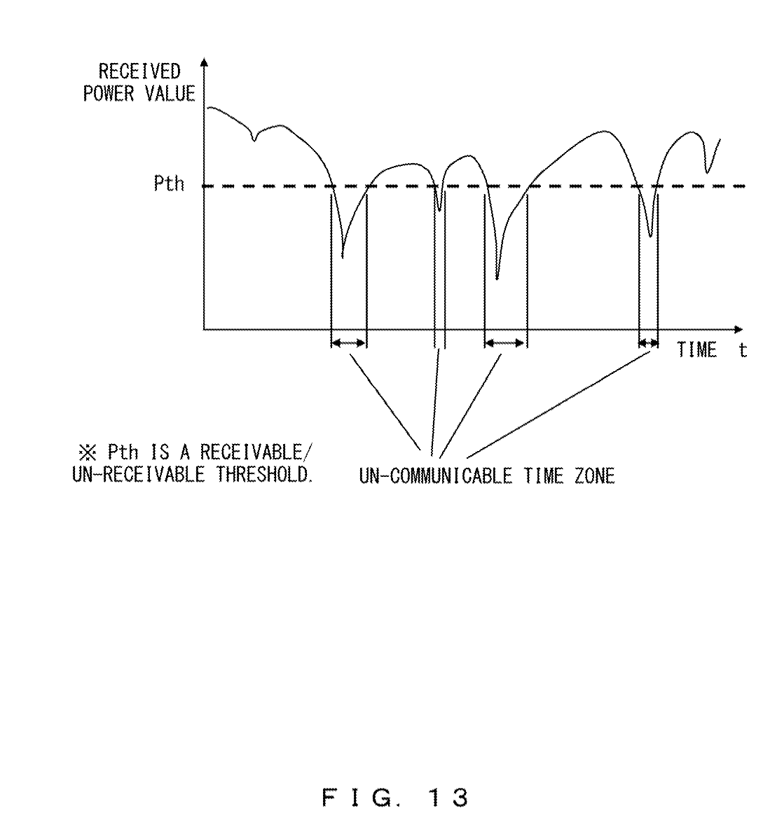

However, in a communication system using electromagnetic waves propagating in the air or ultrasonic waves propagating on a living body surface, the propagation environment of a specific frequency used in the system is affected by a surrounding environment, a near-by object, the interference of another communication system, and the like. As a result, fading as illustrated in FIG. 13 is caused and a time zone in which received power necessary for a communication cannot be secured and communication cannot be conducted is caused.

As a technique for solving this, a diversity technique is known. By this technique, frequencies different from each other are used for respective propagation paths and a correlation between propagation paths is reduced. Simultaneously, propagation states are appropriately monitored and a communication is switched over to propagation paths having better communication quality. Thus, the frequency of communication disconnection can be reduced.

In addition, a technique for conducting data communications in the water using ultrasonic waves is also studied.

However, the diversity technique for avoiding fading has the following two problems when electromagnetic waves are used for all communication paths.

As the first problem there is a possibility that a communication will not be able to be conducted by part of a living body, an obstacle existing in the neighborhood of a living body or the like.

As the second problem there is a possibility that communication content may be eavesdropped on since electromagnetic waves also propagate widely around a living body.

The technique for conducting data communications in the water using ultrasonic waves is specialized for data communications in the water and is not suitable for BAN communications.

Thus, a technique for realizing a stable and high-security communication will be explained below. The preferred embodiments will be explained in detail below.

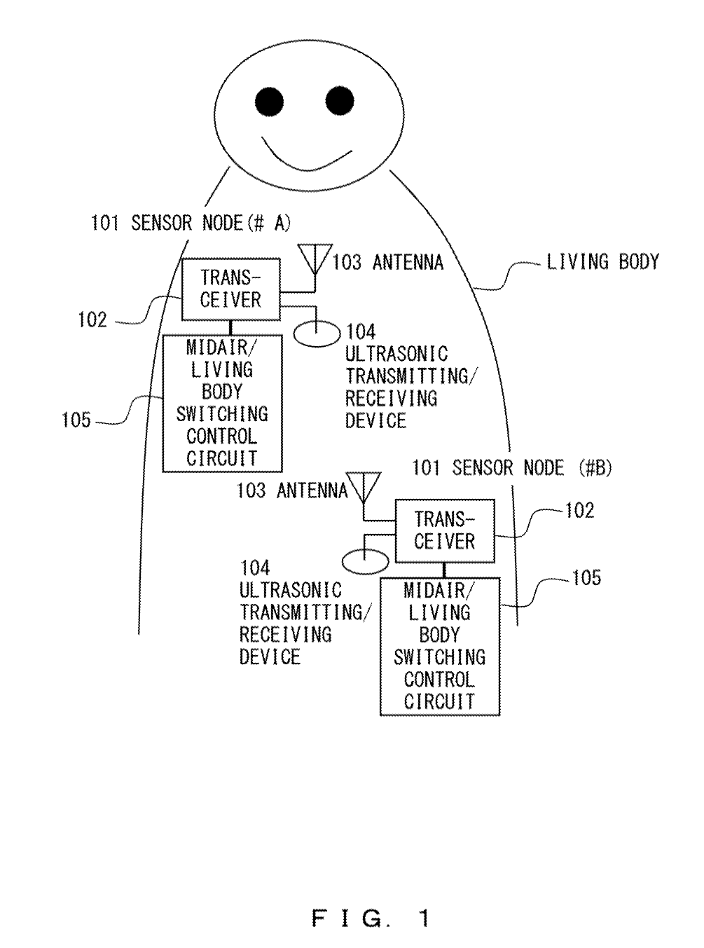

FIG. 1 is a configuration of the preferred embodiment of a sensor network system on a living body. In FIG. 1, sensor nodes 101 are installed on a living body such as a human body (or an animal or the like) in relation to various sensors, for example a blood pressure sensor, a pulse sensor, a heart pacemaker, an electrocardiograph, a blood sugar level meter, or the like. In FIG. 1, only two sensor nodes #1 and #2 are illustrated for the purpose of simplifying the description.

Respective sensor nodes 101 are connected to a transceiver 102, and include an antenna 103 and an ultrasonic transmitting/receiving device 104. The antenna 103 is used for a communication via midair propagation using electromagnetic waves. The ultrasonic transmitting/receiving device 104 is used for a communication via intra-living-body propagation using ultrasonic waves. A midair electromagnetic wave communication performed by the antenna 103 and an intra-living-body ultrasonic wave communication performed by the ultrasonic transmitting/receiving device 104 can be switched between by the midair/living-body switching control circuit 105 controlling the transceiver 102. The ultrasonic transmitting/receiving device 104 can be realized by, for example, a piezo-electric device such as an ultrasonic transducer or the like.

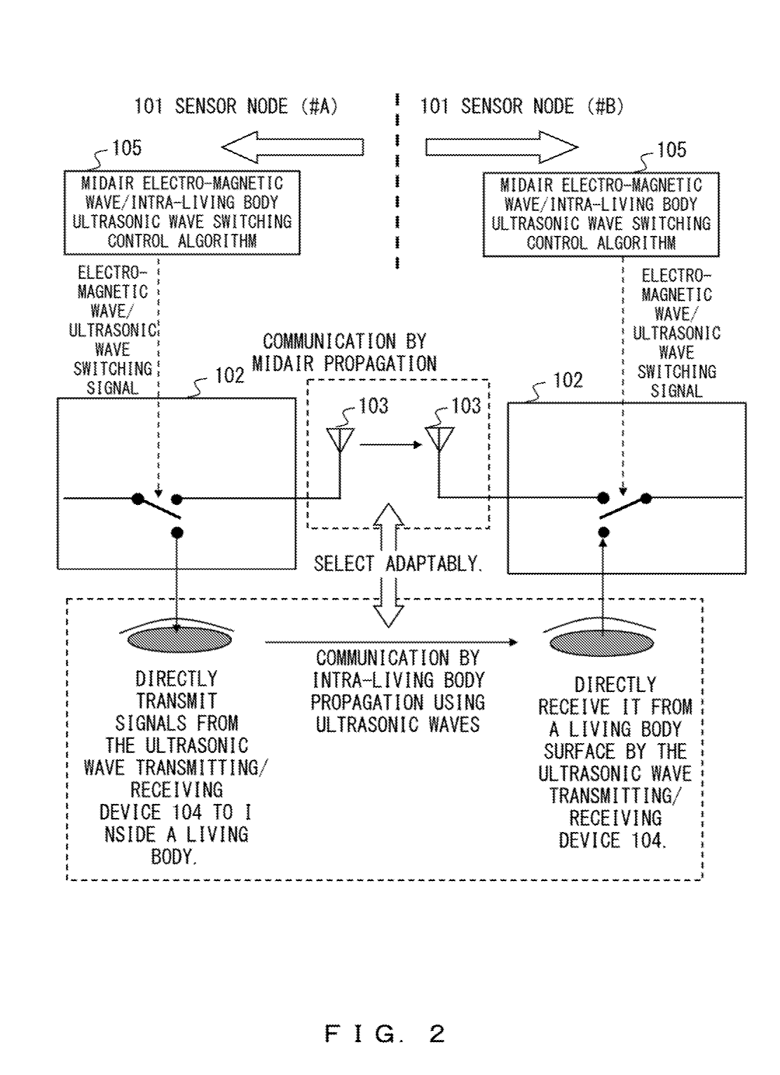

FIG. 2 explains the basic operation of a communication using electromagnetic or ultrasonic waves according to the preferred embodiment illustrated in FIG. 1.

As illustrated in FIG. 2, respective midair electromagnetic wave/intra-living-body ultrasonic wave switching control algorithms operate in the respective midair/living-body switching control circuit 105 of respective sensor nodes 101 #A and #B illustrated in FIG. 1. Respective electromagnetic wave/ultrasonic wave switching signals are output to the transceiver 102 on the basis of this operation.

For example, in the sensor node 101 #A, the midair electromagnetic wave/intra-living-body ultrasonic wave switching control algorithm determines that a communication via midair propagation using electromagnetic waves is suitable. In this case, a switching unit in the transceiver 102 selects a route on the antenna 103 side according to the electromagnetic wave/ultrasonic wave switching signal. As a result, in the sensor node 101 #A, transmitting signals output from the transceiver 102 are transmitted toward the air in the neighborhood of a living body via the antenna 103.

In the midair/living-body switching control circuit 105 of the sensor node 101 #B too, the midair electromagnetic wave/intra-living-body ultrasonic wave switching control algorithm operates on the same basis as in the sensor node 101 #A. As a result, in the sensor node 101 #B, a receiving process is applied to the transmitting signals in the transceiver 102 after they are received by the antenna 103.

However, it is assumed for example that in the sensor node 101 #A, the midair electromagnetic wave/intra-living-body ultrasonic wave switching control algorithm determines that a communication via intra-living-body propagation using ultrasonic waves is suitable. In this case, the switching unit in the transceiver 102 selects a route on the ultrasonic transmitting/receiving device 104 side according to the electromagnetic wave/ultrasonic wave switching signal. As a result, in the sensor node 101 #A, transmitting signals output from the transceiver 102 are transmitted toward a living body via the ultrasonic transmitting/receiving device 104.

In the midair/living-body switching control circuit 105 of the sensor node 101 #B too, the midair electromagnetic wave/intra-living-body ultrasonic wave switching control algorithm operates on the same basis as in the sensor node 101 #A. As a result, in the sensor node 101 #B, a receiving process is applied to the transmitting signals in the transceiver 102 after they are received by the ultrasonic transmitting/receiving device 104.

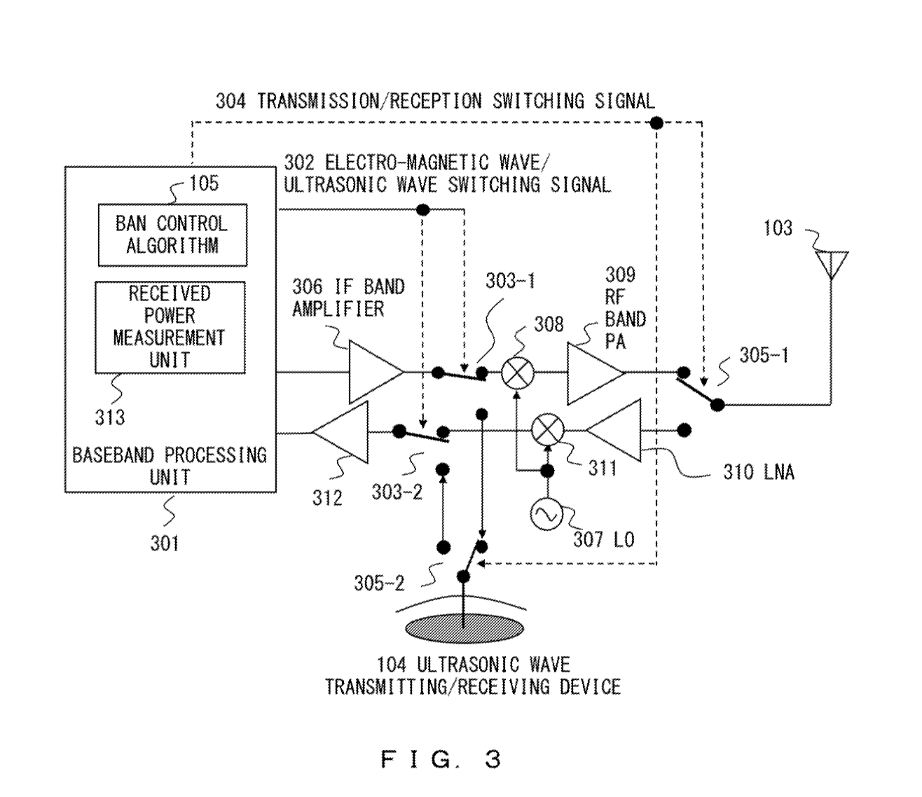

FIG. 3 is a configuration example of the sensor node 101 illustrated in FIG. 1.

In FIG. 3, a baseband processing unit 301 includes a receiving power measurement unit 313 for measuring received power in addition to the midair/living-body switching control circuit 105.

An IF band amplifier (intermediate frequency band amplifier) 306, a multiplier 308 and an RF band PA (radio frequency band power amplifier) 309, an LNA (low noise amplifier) 310, a multiplier 311, an IF band amplifier 312, an LO (local oscillator) 307, electromagnetic wave/ultrasonic wave switching control units 303-1 and 303-2, and transmission/reception switching units 305-1 and 305-2 correspond to the respective transceivers 101 illustrated in FIG. 1. The IF band amplifiers 306 and 312 amplify the transmitting and received signals, respectively, in an intermediate frequency band. The multipliers 308 and 311 convert signals from an intermediate frequency band to a radio frequency band and from a radio frequency band to an intermediate frequency band, respectively, by multiplying transmitting signals and received signals, respectively, by a local oscillation signal from the LO 307. The RF band PA 309 and the LNA 310 amplify transmitting signals and received signals, respectively, in a radio frequency band.

Electromagnetic wave/ultrasonic wave switching signals 302 are supplied from the midair/living-body switching control circuit 105 in the baseband processing unit 301 to the electromagnetic wave/ultrasonic wave switching units 303-1 and 303-2. Transmission/reception switching signals 304 are supplied from the baseband processing unit 301 to the transmission/reception switching units 305-1 and 305-2. The antenna 103 is the same as that illustrated in FIG. 1.

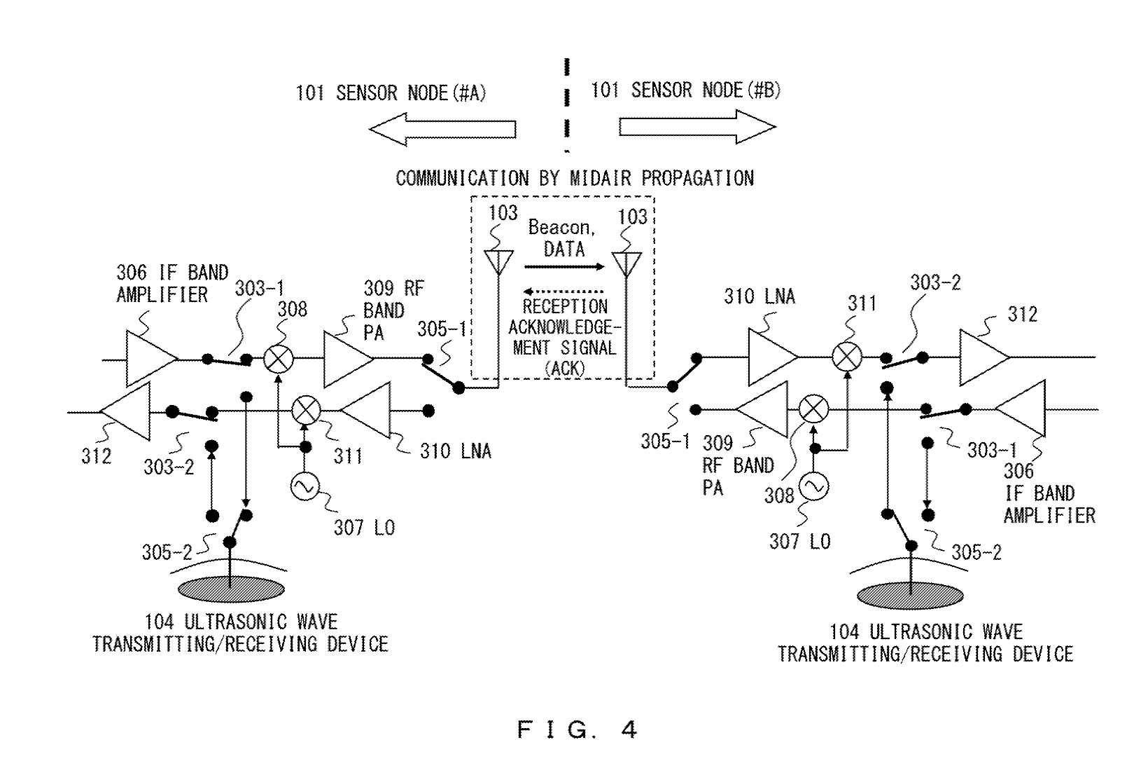

FIG. 4 explains the operation between the sensor nodes 101 having the configuration illustrated in FIG. 3 in the case where a communication via midair propagation using electromagnetic waves is conducted.

In FIG. 4, for example, it is assumed that the sensor nodes 101 #A and #B operate as a transmitting side node and a receiving side node, respectively.

In this case, firstly, in the sensor node 101 #A, the transmission/reception switching unit 305-1 connects the RF band PA 309 and the antenna 103 using the transmission/reception switching signal 304 (see FIG. 3) from the baseband processing unit 301. However, in the sensor node 101 #B, the transmission/reception switching unit 305-1 connects the antenna 103 and the LNA 310 using the transmission/reception switching signal 304 (see FIG. 3) from the baseband processing unit 301.

Then, in the sensor node 101 #A for example, when determining that a communication via midair propagation using electromagnetic waves is suitable, the midair/living-body switching control circuit 105 in the baseband processing unit 301 outputs an electromagnetic wave/ultrasonic wave switching signal 302 for switching the electromagnetic wave/ultrasonic wave switching units 303-1 and 303-2 over to the antenna 103 side.

As a result, in the sensor node 101 #A illustrated in FIG. 4, transmitting signals output from the baseband processing unit 301 in FIG. 3 are transmitted via the following route. Specifically, the transmitting signals are transmitted from the antenna 103 to the air in the neighborhood of a living body via the IF band amplifier 306, the electromagnetic wave/ultra wave switching unit 303-1, the multiplier 308, the RF band PA 309, and the transmission/reception switching unit 305-1.

In FIG. 4, in the sensor node 101 #B too, the midair electromagnetic wave/intra-living-body ultrasonic wave switching control algorithm operates in the midair/living-body switching control circuit 105 in the baseband processing unit 301 on the same basis as in the sensor node 101 #A. Thus, the midair/living-body switching control circuit 105 outputs an electromagnetic wave/ultrasonic wave switching signal 302 for switching the electromagnetic wave/ultrasonic wave switching units 303-1 and 303-2 over to the antenna 103 side.

As a result, in the sensor node 101 #B illustrated in FIG. 4, communication signals received from the sensor node 101 #A by the antenna 103 are received via the following route. Specifically, a receiving process is applied to receiving signals by the baseband processing unit 301 via the transmission/reception switching unit 305-1, the LNA 310, the multiplier 311, the electromagnetic wave/ultrasonic wave switching unit 303-2 and the IF band amplifier 312.

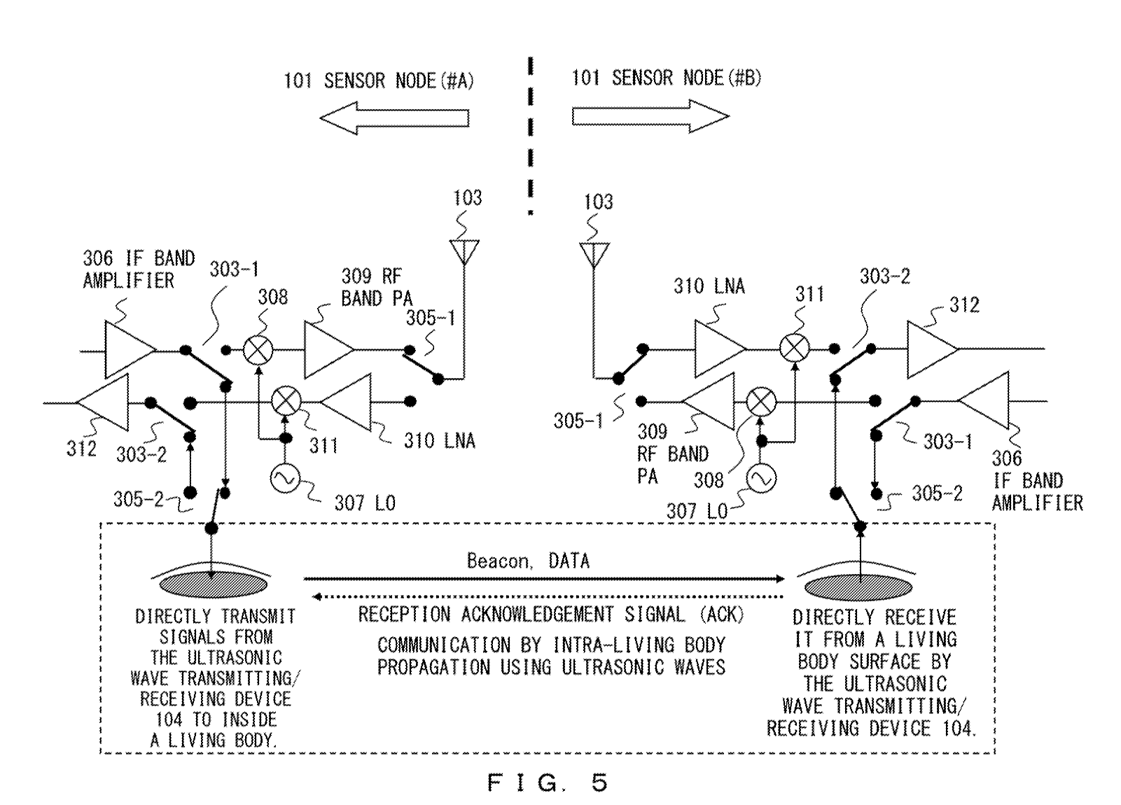

FIG. 5 explains the operation of the preferred embodiment in the case where a communication via intra-living-body propagation using ultrasonic waves is conducted.

In FIG. 5 it is assumed that the sensor nodes 101 #A and #B operate as transmitting and receiving side nodes, respectively.

In this case, firstly, in the sensor node 101 #A, the transmission/reception switching unit 305-2 connects the IF band amplifier 306 and the ultrasonic wave transmitting/receiving device 104 by the transmission/reception switching signal 304 (see FIG. 3) from the baseband processing unit 301. However, in the sensor node 101 #B, the transmission/reception switching unit 305-2 connects the ultrasonic wave transmitting/receiving device 104 and the IF band amplifier 312 by the transmission/reception switching signal 304 (see FIG. 3) from the baseband processing unit 301.

Then, for example, in the sensor node 101 #A it is assumed that the midair/living-body switching control circuit 105 in the baseband processing unit 301 determines that a communication via intra-living-body propagation using ultrasonic waves is suitable. In this case, the midair/living-body switching control circuit 105 outputs the electromagnetic wave/ultrasonic wave switching signal 302 for switching the electromagnetic wave/ultrasonic wave switching units 303-1 and 303-2 over to the ultrasonic wave transmitting/receiving device 104 side.

As a result, in the sensor node 101 #A illustrated in FIG. 5, transmitting signals output from the baseband processing unit 301 illustrated in FIG. 3 are transmitted via the following route. Specifically, the transmitting signals are transmitted from the ultrasonic wave transmitting/receiving device 104 into a living body via the IF band amplifier 306, the electromagnetic wave/ultra wave switching unit 303-1, and the transmission/reception switching unit 305-2.

In FIG. 5, in the sensor node 101 #B as well, the midair electromagnetic wave/intra-living-body ultrasonic wave switching control algorithm operates in the midair/living-body switching control circuit 105 in the baseband processing unit 301 on the same basis as in the sensor node 101 #A. Thus, the midair/living-body switching control circuit 105 outputs an electromagnetic wave/ultrasonic wave switching signal 302 for switching the electromagnetic wave/ultrasonic wave switching units 303-1 and 303-2 over to the ultrasonic wave transmitting/receiving device 104 side.

As a result, in the sensor node 101 #B illustrated in FIG. 5, communication signals received from the sensor node 101 #A by the ultrasonic wave transmitting/receiving device 104 are received via the following route. Specifically, a receiving process is applied to receiving signals by the baseband processing unit 301 illustrated in FIG. 3 via the transmission/reception switching unit 305-2, the electromagnetic wave/ultrasonic wave switching unit 303-2, and the IF band amplifier 312.

Thus, at the time of intra-living-body communication via ultrasonic waves, a communication is conducted between the intermediate frequency processing unit (IF unit) of the IF band amplifiers 306 and 312, and the ultrasonic wave transmitting/receiving device 104 without passing through a radio frequency processing unit (RF unit) such as the multipliers 308 and 311, the RF band PA 309, the LNA 310, the LO 307 and the like.

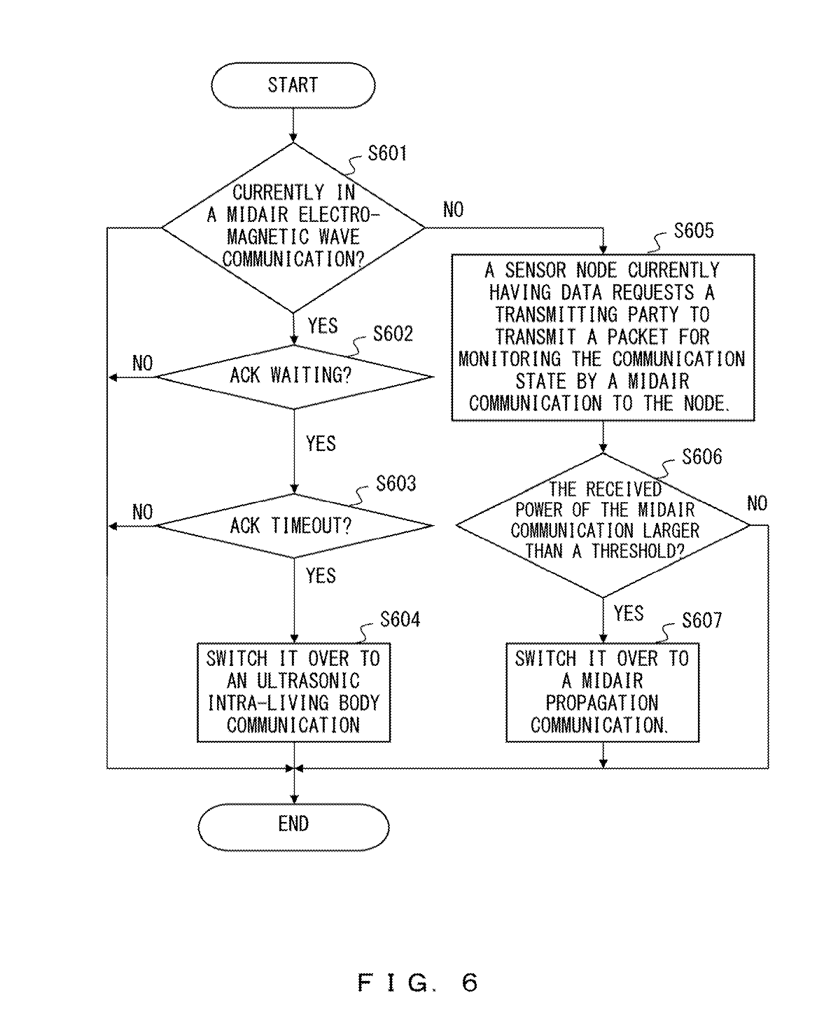

FIG. 6 is an operational flowchart illustrating a midair electromagnetic wave/intra-living-body ultrasonic wave switching control algorithm executed by the midair/living-body switching control circuit 105 at certain time intervals (No. 1).

Firstly, it is determined whether a communication via electromagnetic waves is currently being conducted (step S601).

If a communication via electromagnetic waves is currently being conducted and the determination at step S601 is YES, it is determined whether ACK, which is a confirmative reception confirm response from the sensor node 101 on the opposite party side, is currently being awaited (step S602).

If the ACK is not currently being awaited (NO in step S602), the current process of the operational flowchart illustrated in FIG. 6 terminates.

If the ACK is currently being awaited (YES in step S602), then it is determined whether an ACK waiting time-out has occurred (step S603).

If an ACK waiting time-out has not occurred (NO in step S603), the current process of the operational flowchart illustrated in FIG. 6 terminates.

If an ACK waiting time-out has occurred (YES in step S603), it is determined that the route state of electromagnetic wave communication is bad and the electromagnetic wave communication is immediately switched over to an ultrasonic intra-living-body communication. As described earlier, this switching process is a process of controlling the electromagnetic wave/ultrasonic wave switching signal 302 (see FIG. 5) so as to enable the electromagnetic wave/ultrasonic wave switching units 303-1 and 303-2 to connect the IF band amplifier 306 or 312 with the ultrasonic wave transmitting/receiving device 104 side. Then, the current process of the operational flowchart illustrated in FIG. 6 terminates.

However, if a communication via electromagnetic waves is currently being conducted and the determination at step S601 is NO, the following process is performed.

Firstly, a packet for requesting that a sensor node 101 on an opposite party side (receiving side) transmit a packet via midair communication, for monitoring its communication state, is transmitted from a sensor node 101 currently having data to the sensor node 101 on an opposite party side (receiving side) (step S605). This communication is an intra-living-body communication using ultrasonic waves. As a result, in the sensor node 101 on the opposite party side, the electromagnetic wave/ultrasonic wave switching units 303-1 and 303-2 and the transmission/reception switching units 305-1 and 305-2 are controlled. Thus, a packet signal for monitoring using midair propagation by electromagnetic waves is regularly transmitted from the antenna 103. In the sensor node 101 on its own side, the electromagnetic wave/ultrasonic wave switching units 303-1 and 303-2 and the transmission/reception switching units 305-1 and 305-2 are controlled. Thus, a packet signal for monitoring using midair propagation by electromagnetic waves is regularly received by the antenna 103. Then, the received power of the packet for monitoring is measured by a received power measurement unit 313 in the baseband processing unit 301.

Then, it is determined whether the received power of the midair communication is larger than a threshold value (step S606).

If the received power of the midair communication is not larger than a threshold value and the determination in step S606 is NO, the current process of the operational flowchart illustrated in FIG. 6 terminates.

If the received power of the midair communication is larger than a threshold value and the determination in step S606 is YES, it is determined that the state of an electromagnetic wave communication route becomes good and immediately the communication is switched over to a midair communication via electromagnetic waves (step S607). As described earlier, this switching process is a process of controlling the electromagnetic wave/ultrasonic wave switching signal 302 (see FIG. 5) to enable the electromagnetic wave/ultrasonic wave switching units 303-1 and 303-2 to connect the IF band amplifier 306 or 312 with the antenna 103 side. Then, the current process of the operational flowchart illustrated in FIG. 6 terminates.

As described above, it is because a midair communication via electromagnetic waves has a better communication efficiency when an ultrasonic wave communication is conducted at the minimum possible rate of communication that a communication is attempted to be switched over by a monitor packet to a midair communication via electromagnetic communication at the time of an intra-living-body communication via ultrasonic waves.

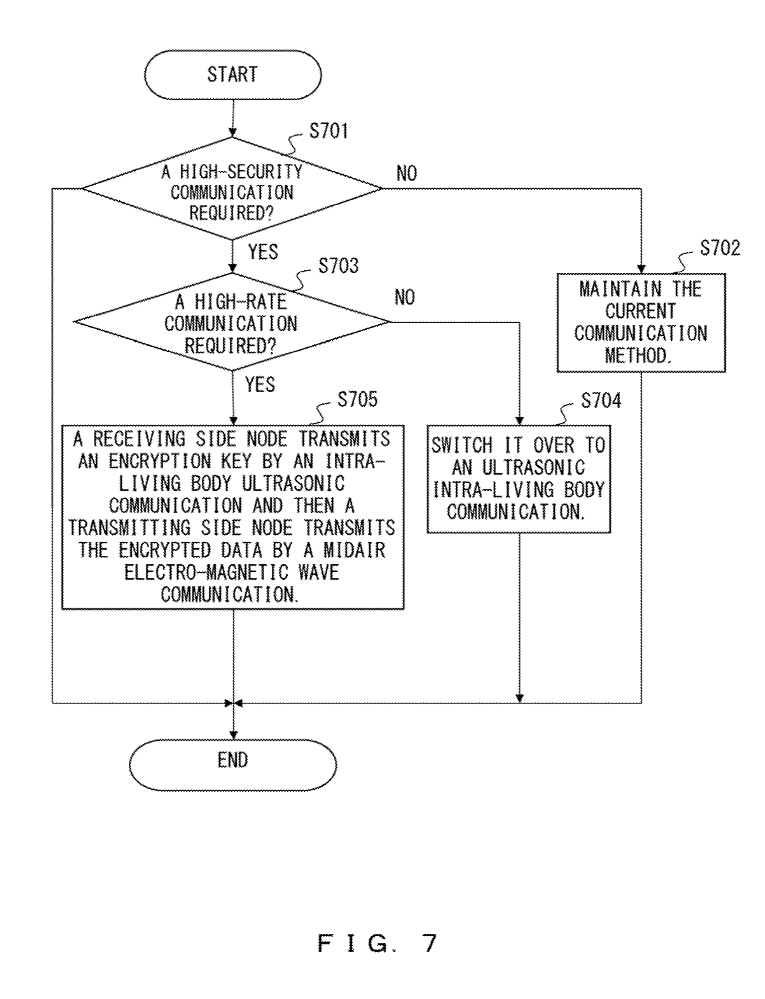

FIG. 7 is an operational flowchart illustrating a midair electromagnetic wave/intra-living-body ultrasonic wave switching control algorithm performed at certain time intervals by the midair/living body switching control circuit 105 when a high security is required (No. 2). This algorithm can be executed independently of (in parallel with) the algorithm illustrated in the operational flowchart of FIG. 6.

Firstly, it is determined whether a high-security communication is required (step S701). This is determined, for example, by the type of a living body sensor used in the sensor node 101 on the transmitting side. For example, the security is high when an electrocardiogram or a sphygmomanometer is connected to the sensor node 101, since an electrocardiogram and blood pressure have characteristic specifying personal information. However, a clinical thermometer and the like have low security.

If a high-security communication is not required and the determination in step S701 is NO, the current communication method is kept (step S702) and the current process of the operational flowchart illustrated in FIG. 7 terminates.

If a high-security communication is required and the determination in step S701 is YES, then it is determined whether a high-rate communication is required (step S703). This is determined, for example, on the basis of whether a real-time communication is required, whether a large number of communications are required, or the like.

If a high-security communication is not required and the determination in step S703 is NO, the communication is switched over to an ultrasonic intra-living-body communication. Then, the current process of the operational flowchart illustrated in FIG. 7 terminates.

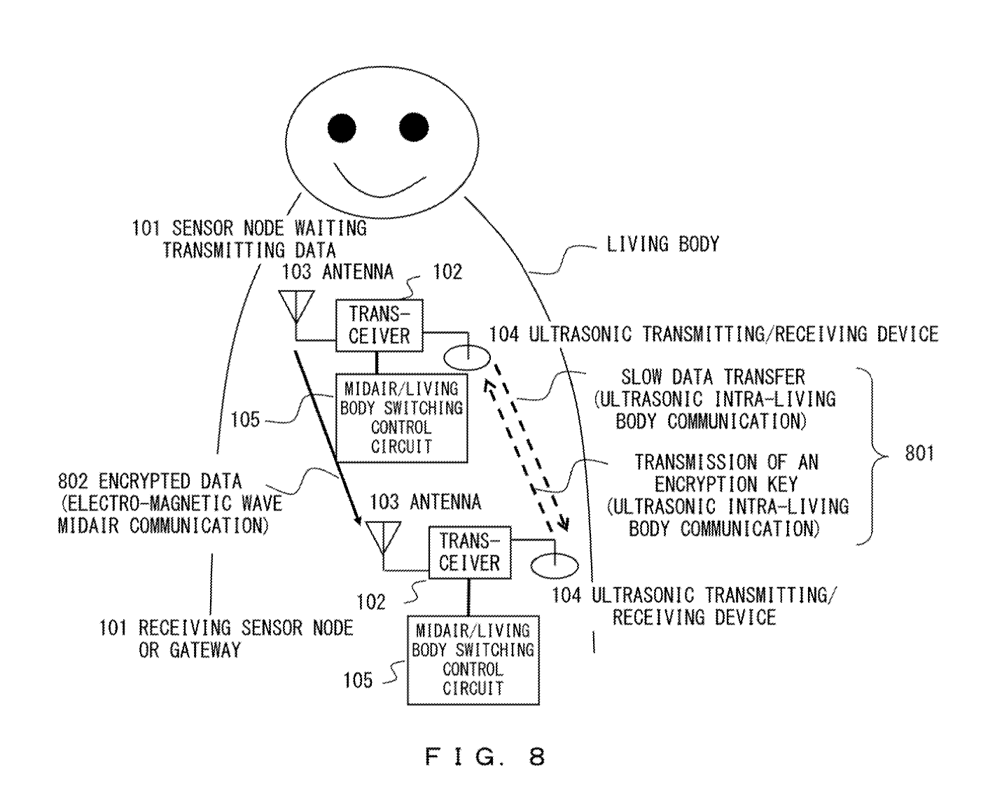

If a high-security communication is required and the determination in step S703 is YES, firstly, as illustrated as 801 in FIG. 8, the communication is switched over to an ultrasonic wave intra-living-body communication and encryption key data is exchanged between the sensor nodes 101 by the intra-living-body communication. Then, as illustrated as 802 in FIG. 8, the communication is switched over to an electromagnetic midair communication. Then, the encryption of communication data using the key data is instructed (step S705). Then, the current process of the operational flowchart illustrated in FIG. 7 terminates. As a result, after that, an encryption communication via electromagnetic wave midair propagation using the exchanged key data is conducted.

Since in an intra-living-body communication via ultrasonic waves there is very little possibility that communication signals will leak out of a living body, ultrasonic waves are suited to transmit important information; however, their transfer rate is low. In contrast, a midair communication via electromagnetic waves is suited to transmit a large number of signals, but it is not a safe transmission path as it is. Therefore, in this preferred embodiment, when a high-security and high-rate communication is required, firstly encryption key data is exchanged between the sensor nodes 101 by an intra-living-body communication via ultrasonic waves. Then, an encryption communication via electromagnetic midair propagation is conducted using this piece of exchanged key data. Thus, a safe and highly efficient BAN communication is made possible.

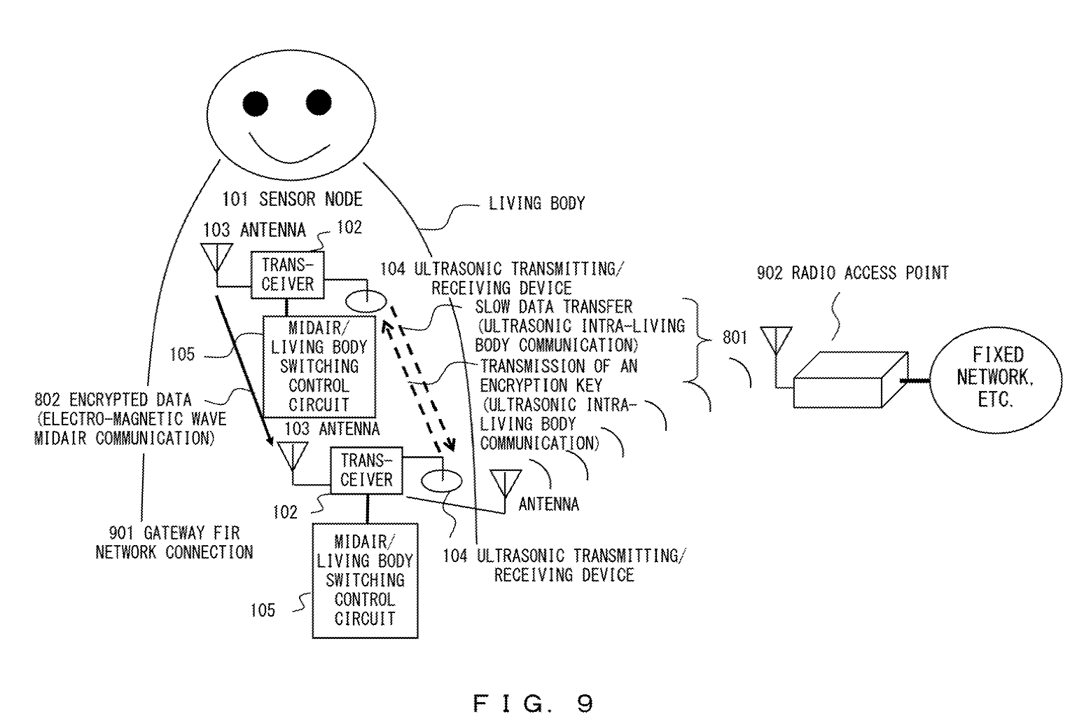

As an application example of a BAN system, an application in which living body data and the like are monitored/analyzed in a medical facility is considered. In this case, it is necessary that an information transmission from the BAN system in the living body to an external network. In this case, for example, as illustrated in FIG. 9, firstly, living body information is transmitted from the sensor node 101 to a gateway 901 for network connection, and then is wirelessly transmitted from there to a radio access point 902 in the neighborhood of the gateway 901. In this case too, since communication data between the sensor node 102 and the gateway 901 is raw data, high security is required. The same procedure based on the operational flowchart illustrated in FIG. 7 as explained in FIG. 8 can be adopted as the procedure of an encryption communication in this case as well.

Thus, in a BAN system in which a connection to a fixed network or the like is assumed as well, high safety and high efficiency can be secured.

In this case, an encryption key is considered for use so as to realize low power consumption in order to suppress the power consumption of the sensor node 101 to a low level.

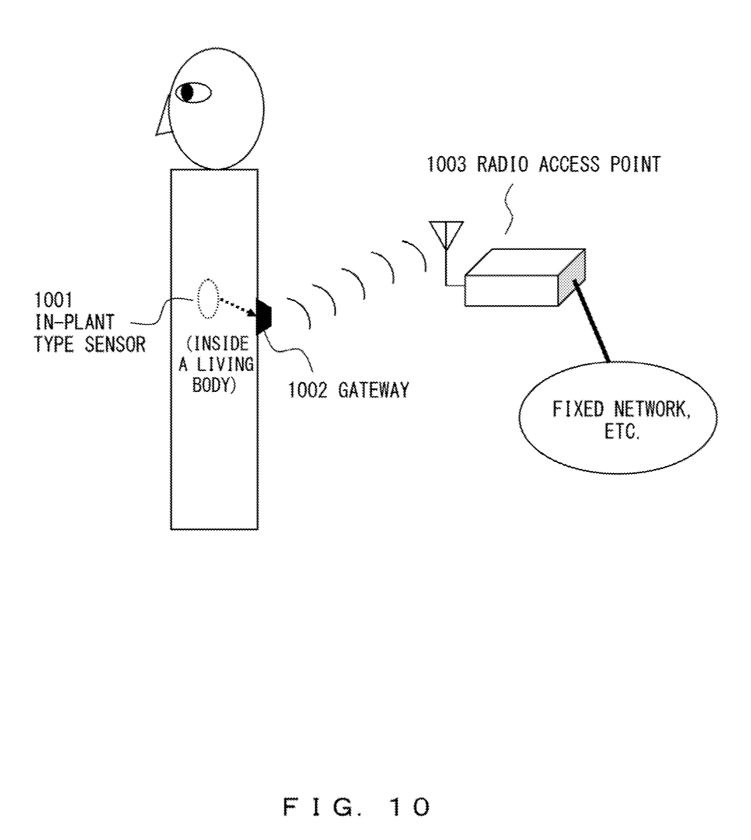

FIG. 10 explains the preferred embodiment using an implant (built-into-body) type sensor. For this preferred embodiment, the same configuration as illustrated in FIG. 9 can be adopted. Specifically, in FIG. 10 an implant type sensor 1001 corresponds to the sensor node 101 illustrated in FIG. 9. A gateway 1002 corresponds to the gateway 901 for network connection illustrated in FIG. 9. A radio access point 1003 corresponds to the radio access point 902 illustrated in FIG. 9.

Thus, a highly safe and highly efficient BAN system using an implant type sensor can also be realized.

In this case, a method for switching between an electromagnetic midair communication and an ultrasonic wave intra-living-body communication mainly led by the midair/living body switching control circuit 105 in the sensor node 101 on the transmitting side can also be adopted. In this case, switching information on the transmitting side is reported from the sensor node 101 on the transmitting side to the sensor node on the receiving side as control information and the midair/living body switching control circuit 105 in the sensor node 101 on the receiving side performs a switching process on the basis of the notice.

Alternatively, the midair/living body switching control circuit 105 in the respective sensor nodes 101 on the transmitting and receiving sides can be configured to perform a switching process on the basis of an algorithm for independently determining its switching.

The disclosed technique can be used, for example, in a medical system which requires continuous and highly reliable monitoring.

If in a system using a diversity technique, an ultrasonic wave communication is used for at least one communication path when a plurality of communication paths can be used, a route of ultrasonic waves cannot be affected by the environment change outside a living body. Therefore, the communication method can be further stabilized as compared to a communication method using electromagnetic waves for all the communication routes. Furthermore, since an ultrasonic wave is remarkably attenuated when enters the air from a living body medium, it is very difficult to eavesdrop on an intra-living-body communication in a place away from the user of a BAN. Therefore, when a plurality of communication paths can be used in a system using a diversity technique, an ultrasonic wave is used for at least one of the communication paths. Thus, an encryption key is transmitted from the receiving side to the transmitting side through the route of ultrasonic waves, and then data from the transmitting side is encrypted and the encrypted data is transmitted by electromagnetic waves propagating in the air. As a result, an electromagnetic wave communication which is difficult to eavesdrop on can be realized, thus improving communication safety.

Thus, according to the disclosed technique, a stabler and higher-security communication than that in a diversity system entirely using a communication method using electromagnetic waves can be realized.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a demonstration of superior and inferior aspects of the invention. Although the embodiments of the present invention have been described in detail, it should be understood that various changes, substitutions, and alterations could be made hereto without departing from the spirit and scope of the invention.

What is claimed is:

1. A radio communication device for communicating sensor information, comprising:
an electromagnetic wave communication unit using electromagnetic waves using the air as a propagation path;
an ultrasonic communication unit using ultrasonic waves using an inside of a living body as a propagation path; and
a midair/living-body switching control unit for switching between a communication performed by the electromagnetic wave communication unit and a communication performed by the ultrasonic wave communication unit.

2. The radio communication device according to claim 1, wherein
when the communication performed by the electromagnetic wave communication unit is conducted, the midair/living-body switching control unit switches from the communication performed by the electromagnetic wave communication unit over to the communication performed by the ultrasonic wave communication unit according to a receiving state of a reception confirm signal from a radio communication device on an opposite party.

3. The radio communication device according to claim 1, wherein
when the communication performed by the ultrasonic wave communication unit is conducted, the midair/living-body switching control unit determines a receiving state of a midair electromagnetic wave communication by attempting to conduct the communication performed by the electromagnetic wave communication unit with a radio communication device on an opposite party at a predetermined timing, and when it is determined that the receiving state has become a predetermined state, the midair/living-body switching control unit switches from the communication performed by the ultrasonic wave communication unit over to the communication performed by the electromagnetic wave communication unit.

4. The radio communication device according to claim 1, wherein
when a high-security data transfer is required, the midair/living-body switching control unit switches the communication over to the communication performed by the ultrasonic wave communication unit so as to conduct an encryption key data communication with a radio communication device on an opposite party, then switches the communication over to the communication performed by the electromagnetic wave communication unit so as to conduct an encryption communication using the encryption key data with a radio communication device on an opposite party.

5. The radio communication device according to claim 1, wherein
when a high-security data transfer which does not requires a high transfer rate is required, the midair/living-body switching control unit switches the communication over to the communication performed by the ultrasonic wave communication unit so as to perform the high-security data transfer.

6. A radio communication method for communicating sensor information, comprising
switching between an electromagnetic wave communication performed by an electromagnetic wave communication unit using the air as a propagation path and an ultrasonic communication performed by an ultrasonic wave communication unit using an inside and/or surface of a living body as a propagation path.
